(19) United States
(12) Patent Application Publication
(10) Pub. No.: US 2005/0244961 A1
(43) Pub. Date: Nov. 3, 2005

(54) Cell culture surface

(76) Inventors: Robert Short, Sheffield (GB); Sheila MacNeil, Sheffield (GB)

Correspondence Address: KLARQUIST SPARKMAN, LLP, 121 SW SALMON STREET, SUITE 1600, PORTLAND, OR, 97204, US

(21) Appl. No.: 10/525,639

(22) PCT Filed: Aug. 20, 2003
(86) PCT No.: PCT/GB03/03634

§ 371 Date: Feb. 22, 2005

(30) Foreign Application Priority Data
Aug. 22, 2002 (GB) 0219544.4
Aug. 27, 2002 (GB) 0219777.0

(51) Int. Cl. C12N 5/08

(52) U.S. Cl. 435/366; 435/368

(57) ABSTRACT

The invention relates to a method for culturing mammalian cells which reduces transfer of xenobiotic material to said cells.

The invention relates to a method for culturing mammalian cells; a cell culture substrate comprising a cell culture surface comprising a plasma polymer of an acid monomer and fibroblast feeder cells; and culture vessels and therapeutic vehicles comprising said substrate.

The culturing of eukaryotic cells, for example mammalian cells, has become a routine procedure and cell culture conditions which allow cells to proliferate are well defined. Typically, cell culture of mammalian cells requires a sterile vessel, usually manufactured from plastics, defined growth tnedium and, in some examples, fibroblast feeder cells and serum, typically calf serum. The feeder cells function to provide mitogenic signals which stimulate cell proliferation and/or maintain cells in an undifferentiated state. The feeder cells are typically fibroblasts which have been treated such that the fibroblasts cannot proliferate (e.g. mitomycin or irradiation treatment). Typically, feeder fibroblasts are murine in origin (as in Rheinwald and Green, 1975). It would be advantageous if cell culture conditions could be established which did not require the addition of xenobiotic materials such as bovine serum or murine cells since their use increases the likelihood of infectious agents (e.g. viruses and prions, in particular for bovine products, and murine viruses for mouse feeder cells) infecting mammalian cells grown in culture. With respect to feeder cells it would be advantageous also if autologous fibroblasts could be used as a feeder layer and that these could be growth arrested without the use of mitomycin C or irradiation treatment.

Tissue engineering is an emerging science which has implications with respect to many areas of clinical and cosmetic surgery. More particularly, tissue engineering relates to the replacement and/or restoration and/or repair of damaged and/or diseased tissues to return the tissue and/or organ to a functional state. For example, and not by way of limitation, tissue engineering is useful in the provision of skin grafts to repair wounds occurring as a consequence of: contusions, or burns, or failure of tissue to heal due to venous or diabetic ulcers. Tissue engineering requires in vitro culturing of replacement tissue followed by surgical application of the tissue to a wound to be repaired. To increase the likelihood that the in vitro generated tissue is free from infectious agents it would be desirable to reduce or avoid exposure of tissue to xenobiotic agents which maybe present in serum or xenobiotic cells

We have utilised “plasma polymerisation” to fabricate cell culture vessels for the culture of mammalian cells.

Plasma polymerisation is a technique which allows an ultra-thin (eg ca.200 nm) cross linked polymeric film to be deposited on substrates of complex geometry and with controllable chemical functionality. As a consequence, the surface chemistry of materials can be modified, without affecting the bulk properties of the substrate so treated. Plasmas or ionised gases are commonly excited by means of an electric field. They are highly reactive chemical environments comprising ions, electrons, neutrals (radicals, metastables, ground and excited state species) and electromagnetic radiation. At reduced pressure, a regime may be achieved where the temperature of the electrons differs substantially from that of the ions and neutrals. Such plasmas are referred to as “cold” or “non-equilibrium” plasmas. In such an environment many volatile organic compounds (eg volatile alcohol containing compounds, volatile acid containing compounds, volatile amine containing compounds, or volatile hydrocarbons, neat or with other gases, eg Ar, have been shown to polymerise (H. K. Yasuda, Plasma Polymerisation, Academic Press, London 1985) coating both surfaces in contact with the plasma and those downstream of the discharge. The organic compound is often referred to as the “monomer”. The deposit is often referred to as “plasma polymer”. The advantages of such a mode of polymerisation potentially include: ultra-thin pin-hole free film deposition; plasma polymers can be deposited onto a wide range of substrates; the process is solvent free and the plasma polymer is free of contamination. Under conditions of low power, plasma polymer films can be prepared which retain a substantial degree of the chemistry of the original monomer. For example, plasma polymerised films of acrylic acid contain the carboxyl group. The low power regime may be achieved either by lowering the continuous wave power, or by pulsing the power on and off.

Co-polymerisation of one or more compounds having functional groups with a hydrocarbon allows a degree of control over surface functional group concentrations in the resultant plasma copolymer (PCP). Suitably, the monomers are ethylenically unsaturated. Thus the functional group compound maybe unsaturated carboxylic acid, alcohol or amine, for example, whilst the hydrocarbon is suitably an alkene.

By plasma polymerisation, it is also possible to deposit ethylene oxide-type molecules (eg. tetraethyleneglycol monoallyl ether) to form ‘non-fouling’ surfaces. It is also possible to deposit perfluoro-compounds (i.e. perfluorohexane, hexafluoropropylene oxide) to form hydrophobic/superhydrophobic surfaces. This technique is advantageous because the surfaces have unique chemical and physical characteristics. Moreover, the surface wettability, adhesion and frictional/wear characteristics of the substrate can be modified in a controllable and predictable manner.

In WO00/78928 we disclose a therapeutic vehicle which comprises a surface with high acid functionality which is obtainable by the method of plasma polymerisation. (high acid functionality describes the high degree of carboxyl (acid) retention achieved from the monomer in plasma polymerisation; not the amount of acid in the surface). The vehicle treated in this way provides a structure which can support the attachment and proliferation of cells and importantly the detachment of cells to invade and repair a wound bed. In WO03/035850 we disclose a polymeric substrate comprising a surface with high acid functionality which is also obtainable by plasma polymerisation which has utility in the delivery of cells to a wound in need of repair.

The present invention relates to a cell culture vessel which is treated by plasma polymerisation and surprisingly has interesting properties with respect to cell culture conditions required to maintain cells in culture in the absence of serum. The invention also relates to a method to culture cells on therapeutic vehicles which are subsequently used in the repair of damaged tissue.

According to an aspect of the invention there is provided a method for the culture of mammalian cells comprising the steps of:
i) providing a cell culture vessel comprising:
   a) mammalian cells;
   b) a cell culture support comprising a substrate wherein said substrate comprises a cell culture surface wherein said surface comprises a polymer of an acid monomer and attached thereto, fibroblast feeder cells
   c) cell culture medium sufficient to support the growth of said mammalian cells wherein said medium does not include serum; and
ii) providing cell culture conditions which promote the proliferation of said mammalian cells.

In a preferred method of the invention said mammalian cells are human.

In a further preferred method of the invention said mammalian cells are maintained in culture in an undifferentiated state. Preferably said cells are undifferentiated keratinocytes, for example keratinocyte stem cells.

Advantageously, the method according to the invention allows the maintenance of certain cell-types, e.g. keratinocytes, in an undifferentiated state for extended peroids of culture thereby allowing continued cell expansion and the production of substantial amounts of cellular material which can be used in clinical applications, for example the repair of wounds.

In a further preferred method of the invention said mammalian cells are selected from the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

The invention includes other combinations of cells which in vivo act as support cells supplying a trophic signals to more specialised differentiated cells. A further example of this would be autologous cells, e.g. fibroblasts or epithelial cells acting as a feeder layer to support the survival and expansion of cancer cells required for the diagnosis or treatment of patients-e.g. when tumour cells are cultured with cells of the immune system under conditions designed to induce a host immune response when cells (eg tumour infiltrating lymphoctes) are reintroduced to the patient

In a preferred method of the invention said mammalian cells are keratinocytes, preferably autologous keratinocytes.

Typically, mammalian cells, or as illustrated in the examples, keratinocytes are seeded at a cell density of about 0.75×10⁴ cells/mm². This is shown to work well and provides good surface coverage, see Table 3.

In a preferred method of the invention the number of said mammalian cells and said fibroblast cells in co-culture is at a ratio of about between 1:1-5:1 (mammalian cell:fibroblast cell). Preferably said ratio is about 5:1.

Preferably said mammalian cells are keratinocytes and are in a ratio of about 5:1 with said fibroblast cells.

If the ratio of mammalian cells, for example keratinocytes, to fibroblast feeder cells is about 1:5, the fibroblast cells do not need to be lethally irradiated but can be used in a proliferative state.

In a further preferred method of the invention said vessel is selected from the group consisting of: a petri-dish; cell culture bottle or flask; multiwell plate. “vessel” is construed as any means suitable to contain a mammalian cell culture.

In a preferred method of the invention said substrate comprises a non-porous polymer. Preferably a solid-phase substrate, e.g. plastics, glass, contact lenses.

Plasma coating of porous and fibrous materials, woven and non-woven materials, are also within the scope of the invention (e.g. bandages, gauze, plaster casts).

Plastics used in the manufacture of cell culture vessel products include polyethylene terephthalate, high density polyethylene, low density polyethylene, polyvinyl chloride, polypropylene or polystyrene.

In a preferred method of the invention said cell culture surface comprises a polymer comprising an acid content of at least 2%. Preferably said acid content is 2-20%. Alternatively said acid content is greater than 20%. The percentages refer to the percent of carbon atoms in this type of environment. For example 20% acid means that 20 of every one hundred carbons in the plasma polymer is in an acid type environment. The acid content of a cell culture surface is determined by methods herein disclosed and are known in the art. For example, percent acid maybe measured by x-ray photoelectron spectroscopy.

Polymerizable monomers that may be used in the practice of the invention preferably comprise unsaturated organic compounds such as halogenated olefins, olefinic carboxylic acids and carboxylates, olefinic nitrile compounds, olefinic amines, oxygenated olefins and olefinic hydrocarbons. Such olefins include vinylic and allylic forms. The monomer need not be olefinic, however, to be polymerizable. Cyclic compounds such as cyclohexane, cyclopentane and cyclopropane are commonly polymerizable in gas plasmas by glow discharge methods. Derivatives of these cyclic compounds, such as 1,2- diaminocyclohexane for instance, are also commonly polymerizable in gas plasmas. Particularly preferred are polymerizable monomers containing hydroxyl, amino or carboxylic acid groups. Of these, particularly advantageous results have been obtained through use of allylamine or acrylic acid. Mixtures of polymerisable monomers may be used. Additionally, polymerisable monomers may be blended with other gases not generally considered as polymerisable in themselves, examples being argon, nitrogen and hydrogen. The polymerisable monomers are preferably introduced into the vacuum chamber in the form of a vapour. Polymerisable monomers having vapour pressures less than 1.3×10⁻² mbar are not generally suitable for use in the practice of this invention.

Polymerisable monomers having vapour pressures of at least 6.6×10² mbar at ambient room temperature are preferred. Where monomer grafting to plasma polymerisate deposits is employed, polymerisable monomers having vapor pressures of at least 1.3 mbar at ambient conditions are particularly preferred.

To maintain desired pressure levels, especially since monomer is being consumed in the plasma polymerisations operation, continuous inflow of monomer vapor to the plasma zone is normally practiced. When non-polymerisable gases are blended with the monomer vapour, continuous removal of excess gases is accomplished by simultaneously pumping through the vacuum port to a vacuum source. Since some non-polymerisable gases are often evolved from glow discharge gas plasmas, it is advantageous to control gas plasma pressure at least in part through simultaneous vacuum pumping during plasma polymerisate deposition on a substrate in the process of this invention.

Other examples include, fully saturated and unsaturated carboxylic acid compounds up to 20 carbon atoms. More typically 2-8 carbons. Ethylenically unsaturated compounds (especially α,β unsaturated carboxylic acids) including acrylic acid, methacrylic acid. Saturated including ethanoic acid and propanoic acid. Compounds that can be plasma polymerised that readily hydrolyse to give carboxylic acid functionalities, e.g. organic anhydrides (e.g. maleic anhydride) acyl chlorides.

In a further preferred method of the invention said polymer comprises an acrylic acid monomer with at least 2% acid content. Preferably said acid content is between 2% and 10%. Preferably said acid content is about 4-5% (e.g. 4.5%)

In a further preferred method of the invention said polymer comprises an acid co-polymer. The copolymer is prepared by the plasma polymerisation of an organic carboxylic acid (or anhydride) with a saturated (alkane) or unsaturated (alkene, diene or alkyne) hydrocarbon. The hydrocarbon would be of up to 20 carbons (but more usually of 4-8). Examples of alkanes are butane, pentane and hexane. Examples of alkenes are butene and pentene. An example of a diene is 1-7 octadiene. The comonomer may also be aromatic-containing e.g. styrene.

Co-plasma polymerisation may be carried out using any ratio of acid:hydrocarbon, but will be typically using an acid: hydrocarbon ratio between the limits of 100(acid):0(hydrocarbon) to 20 (acid):80 (hydrocarbon) and any ratio between these limits.

Plasma polymerised amines are also within the scope of the invention, for example, fully saturated primary, secondary or tertiary amines (e.g. butyl amine, propyl amine, heptylamine) or unsaturated e,g, allyl amine, which are at least 20 carbons but more typically 4-8 carbons. Amines could be co-polymerised with hydrocarbons as above.

Plasma polymerised alcohols are also within the scope of this invention, for example allyl alcohol or ethanol. Alcohols could be co-polymerised as above.

The glow discharge through the gas or blend of gases in the vacuum chamber may be initiated by means of an audiofrequency, a microwave frequency or a radiofrequency field transmitted to or through a zone in the vacuum chamber. Particularly preferred is the use of a radiofrequency (RF) discharge, transmitted through a spatial zone in the vacuum chamber by an electrode connected to an RF signal generator. A rather broad range of RF signal frequencies starting as low as 50 kHz may be used in causing and maintaining a glow discharge through the monomer vapor. In commercial scale usage of RF plasma polymerisation, an assigned radiofrequency of 13.56 MHz may be more preferable to use to avoid potential radio interference problems as with examples given later.

The glow discharge need not be continuous, but may be intermittent in nature during plasma polymerisate deposition. Or, a continuous glow discharge may be employed, but exposure of a substrate surface to the gas plasma may be intermittent during the overall polymerisate deposition process. Or, both a continuous glow discharge and a continuous exposure of a substrate surface to the resulting gas plasma for a desired overall deposition time may be employed. The plasma polymerisate that deposits onto the substrate generally will not have the same elemental composition as the incoming polymerisable monomer (or monomers). During the plasma polymerisation, some fragmentation and loss of specific elements or elemental groups naturally occurs. Thus, in the plasma polymerisation of allylamine, nitrogen content of the plasma polymerisate is typically lower than would correspond to pure polyallylamine. Similarly, in the plasma polymerisation of acrylic acid, carboxyl content of the plasma polymerisate is typically lower than would correspond to pure polyacrylic acid. Exposure time to either of these unreacted monomers in the absence of a gas plasma, as through intermittent exposure to a glow discharge, allows for grafting of the monomer to the plasma polymerisate, thereby increasing somewhat the level of the functional group (amine or carboxylic acid) in the final deposit. Time intervals between plasma exposure and grafting exposure can be varied from a fraction of a second to several minutes.

Plasma polymerisation conditions may be adapted by one skilled in the art to atmospheric plasma (i.e without the use of a vacuum plasma reactor)

In a further preferred method of the invention said fibroblast feeder cells are non-proliferative.

In a further preferred method of the invention feeder cells are rendered non-proliferative by a method which avoids the use of mitomycin C or irradiation by lowering the calcium concentration of the medium. For example calcium levels could be provided which enable the grow of mammalian cells in co-culture but inhibit or prevent the growth of feeder cells. Typically, calcium levels could be reduced to about one-tenth physiological levels.

In a further preferred method of the invention said feeder cells are human fibroblasts, preferably human dermal fibroblasts. A further source of feeder cells are oral fibroblasts.

According to a further aspect of the invention there is provided a cell culture vessel comprising: a cell culture support comprising a substrate wherein said substrate comprises a cell culture surface wherein said surface comprises a polymer of an acid monomer and attached thereto, fibroblast feeder cells.

In a preferred embodiment of the invention said fibroblast feeder cells are non-proliferative.

In a preferred embodiment of the invention said vessel further comprises mammalian cells and cell culture medium wherein said medium does not include serum.

In a preferred embodiment of the invention said mammalian cells are selected from the group consisting of: keratinocyte; fibroblast; adult skin stem cell; embryonic stem cell; melanocyte.

In a preferred embodiment of the invention said mammalian cells are keratinocytes, preferably autologous keratinocytes.

According to a further aspect of the invention there is provided a method to treat a cell culture vessel comprising the steps of:
i) providing at least one acid monomer source in a gas feed;
ii) creating a plasma of said acid monomer; and
iii) bringing into contact a cell culture vessel with said plasma monomer to provide a cell culture vessel comprising an acid polymer.

In a preferred method of the invention said acid monomer source comprises 30-99% acid monomer. Preferably said acid monomer source consists of a 100% acid monomer source. Preferably said method consists of a 100% acrylic acid source.

According to a further aspect of the invention there is provided a method to treat a cell culture vessel comprising the steps of:
i) providing a selected ratio of an acid containing monomer and a hydrocarbon in a gas feed;
ii) creating a plasma of said mixture;
iii) bringing into contact a cell culture vessel with said plasma mixture to provide a cell culture surface comprising an acid co-polymer.

In a preferred method of the invention said plasma is created by means of electrical power input (radio frequency 13.56 MHz), coupled by means of a copper coil or bands.

The reactor volume is in the range 2-10 L and the reactor is pumped by means of a double stage rotary pump to a base pressure approaching 10⁻⁴ mbar. In the case of replacing the rotary pump with a turbomolecular pump better base pressures can be achieved. The monomer pressure is in the range 10⁻¹ mbar to 10⁻³ mbar and the monomer flow rate is 1-10 cm³/min. The power would be typically 0.5-50W continuous wave. Those skilled in the art may adjust these parameters to produce like plasmas by pulsing on the micro or milli secod time scales, or by using smaller or larger volume reactors .

According to a yet further aspect of the invention there is provided a method to culture mammalian cells on a therapeutic vehicle comprising the steps of:
i) providing a preparation comprising;
   a) mammalian cells;
   b) a therapeutic vehicle wherein said vehicle comprises a substrate which comprises a surface wherein said surface comprises a polymer of an acid monomer and attached thereto, fibroblast feeder cells;
   c) cell culture medium sufficient to support the growth of said mammalian cells wherein said medium does not include serum; and
ii) providing cell culture conditions which promote the proliferation of said mammalian cells on said therapeutic vehicle.

In a preferred method of the invention said mammalian cells are human.

In a further preferred method of the invention said mammalian cells are selected from the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

Preferabaly said mammalian cells are autologous, preferably autologous keratinocytes.

In a further preferred method of the invention said fibroblast feeder cells are human.

In a further preferred method of the invention said fibroblast feeder cells are human dermal fibroblasts or human oral fibroblasts. Preferably said feeder cells are autologous.

In a preferred method of the invention the number of said mammalian cells and said fibroblast cells in co-culture with said vehicle is at a ratio of about between 1:1-5:1 (mammalian cell:fibroblast cell). Preferably said ratio is about 5:1.

Preferably said mammalian cells are keratinocytes and are in a ratio of about 5:1 with said fibroblast cells.

In a preferred method of the invention said mammalian cells, for example keratinocytes, are seeded at a cell density of for example about 0.75×10⁴ cells/mm².

In a further preferred method of the invention said therapeutic vehicle is selected from the group consisting of: prothesis; implant; matrix; stent; biodegradable matrix; or polymeric film.

In a further preferred method of the invention said therapeutic vehicle comprises a substrate composed of a polymeric material, preferably a vinyl polymer.

Preferably said vinyl polymer is selected from the group consisting of: polyvinyl chloride, polyvinyl acetate, polyvinyl alcohol.

Alternative polymeric materials are available and include the following examples.

Examples of substrates include, olefins which includes polyethylene [PE] (very low density, linear low density, chlorinated (<20%) and aliphatic polyolefins (other than polyethylene)) eg polypropylene (PP) polybut-1-ene (atactic), polyisobutylene and diene rubbers (eg natural rubber, styrene butadiene rubber (incl. latex) butyl rubber, nitrile rubber, polybutadiene, polyisoprene ethylene-propylene rubber, and polychlorprene). PE and other aliphatic olefins may contain additives such as antioxidants, antiozonates, softners, processing aids, blowing agents, pigments, and filers.

Further examples include, ethylene co-polymers such as ethylene vinyl actetate and ethylene ethyl acrylate; vinyl chloride polymers such as polyvinyl chloride, polyvinyl acetate, polyvinyl alcohol which can include stabilisers, plasticizers, lubricants, fillers and miscellaneous additives; acrylics such as acrylic rubbers and acrylic polymer blends; styrene based plastics such as styrene isoprene and styrene thermoplastic elastomers; polyamides such as polyamide 12 and polyamide co-polymers; silicones polymers, an example of which is polydimethyl siloxane, and silicone rubbers; polyurethanes; polyurethane rubbers; and polysulphides.

Further substrates comprises a polymer selected from the group consisting of: the olefins, which includes polyethylene (very low density, linear low density, chlorinated (<20%) and aliphatic polyolefins (other than polyethylene) eg polypropylene (PP) polybut-1-ene (atactic) and polyisobutylene).

Still further substrates comprises a polymer selected from the group consisting of: diene rubbers (eg natural rubber, styrene butadiene rubber (incl. latex) butyl rubber, nitrile rubber, polybutadiene, polyisoprene, ethylene-propylene rubber, polychlorprene.

Substrates which comprises a polymer selected from the group consisting of: ethylene co-polymers such as ethylene vinyl acetate and ethylene ethyl acrylate are within the scope of the invention.

Further examples include substrates comprises a polymer selected from the group consisting of: acrylics such as acrylic rubbers and acrylic polymer blends; styrene based plastics such as styrene isoprene and styrene thermoplastic elastomers; polyamides such as polyamide 12 and polyamide co-polymers; silicone polymers an example of which is polydimethyl siloxane and silicone rubbers; polyurethanes; polyurethane rubbers; and polysulphides; polyvinyl chloride, polypropylene, silicone and polyhydroxybutyrate. Examples include, PVC (PL 1240, Baxter), PVC (PL 146, Baxter), PVC (410 CU, Pall Medical), PVC (5550 Seta, Solmed), PVC (3226 Seta, Solmed), Polypropylene (7210, Solmed), Polyhydroxybutyrate (Goodfellow) and Silicone-poly dimethylsiloxane (Baxter). Substrates which comprise a polymer of a thermoplastic polyurethane formulation of the form polyol reacted with isocyanate and including polyol or polyamine chain extenders are also included as are polymers of thermosetting polyurethane formulation of the form polyol/polyamine crosslinked with an isocyanate or diisocyanate molecule including tolyl diisocyanate or methyl diisocyanate.

Hydrogels are also included within the scope of the invention. Hydrogels are amorphous gels or sheet dressings which are crosslinked and which typically consist of a polymer, a humectant and water in varying ratios. Hydrogels are known in the art and are commercially available. Hydrogels/sheets function to maintain a moist wound environment and can be removed without trauma to a wound bed. Examples of commercially available hydrogels are Tegagel™, Nu-Gel™ or FlexiGel™.

In a preferred method of the invention said therapeutic vehicle is a hydrogel.

The direct culturing of mammalian cells on a therapeutic vehicle under conditions hereindisclosed has obvious benefits in tissue engineering since the fabrication of the surface of said vehicle allows both culturing, implantation and transfer of cells to a wound to be repaired with ease. Plasma polymerisation conditions disclosed herein for the treatment of cell culture vessels are equally applicable to the treatment of the surfaces of therapeutic vehicles.

An embodiment of the invention will now be described by example only and with reference to the following tables and figures:

Table 1 is a summary of XPS results for plasma polymers made from acrylic acid and octadiene at 10W;

Table 2 is a summary of XPS results for plasma polymers made from allyl amine at 10W;

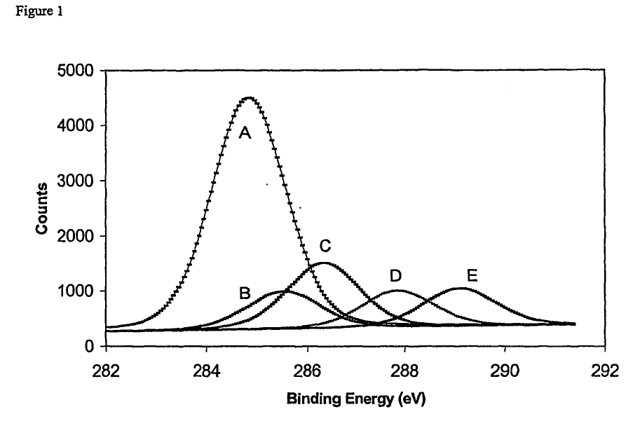

FIG. 1 illustrates peak fitted C 1s core level of plasma polymerised acrylic acid fabricated at 10W. A=C—C/C—H. B=COOH/R (β-shift). C=C—OH/R. D=C=O. E=COOH/R (carboxylate group);

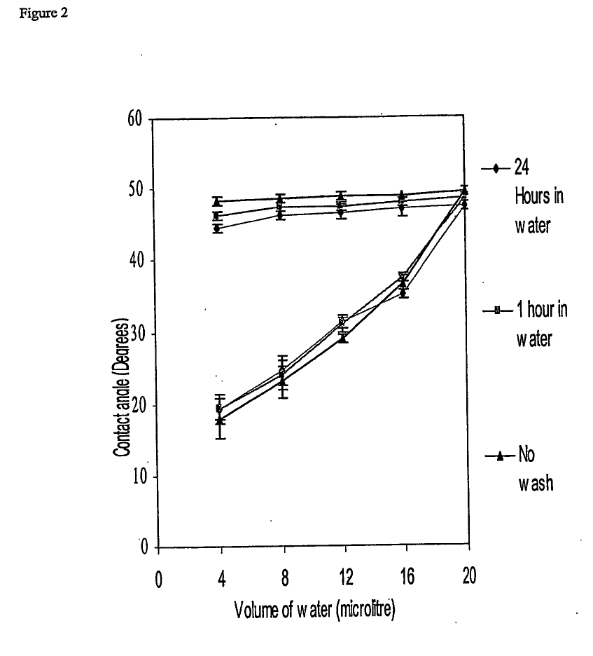

FIG. 2 illustrates advancing and receding contact angle measurements on a pure acrylic acid surface fabricated at 10W following submergence in water for 0, 1 and 24 hours;

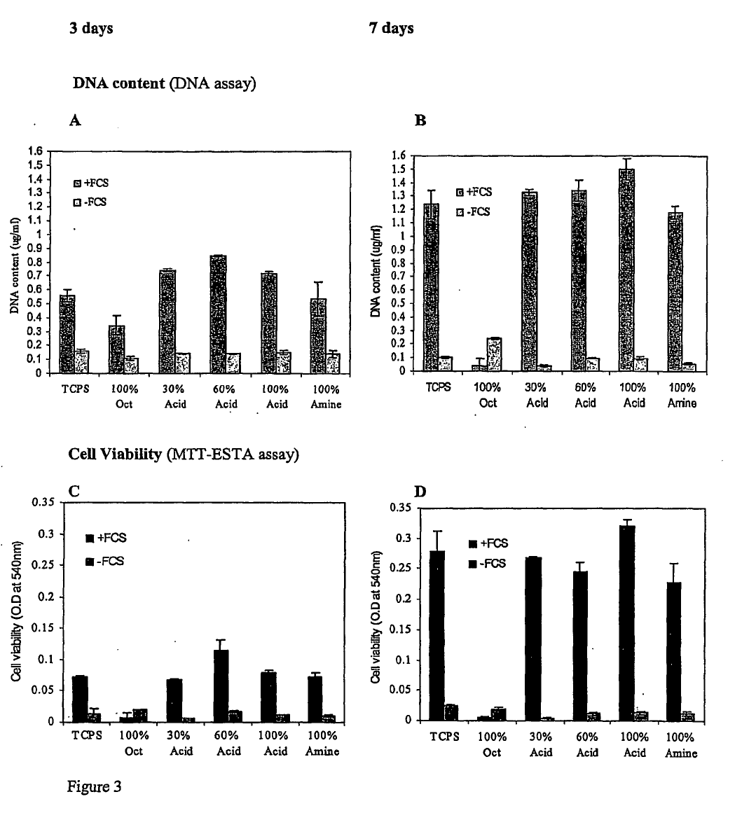

FIG. 3 illustrates proliferation of human dermal fibroblasts on plasma polymer surfaces after 3 and 7 days of culture. Results shown are the means +/− standard deviation of the mean of triplicate cells;

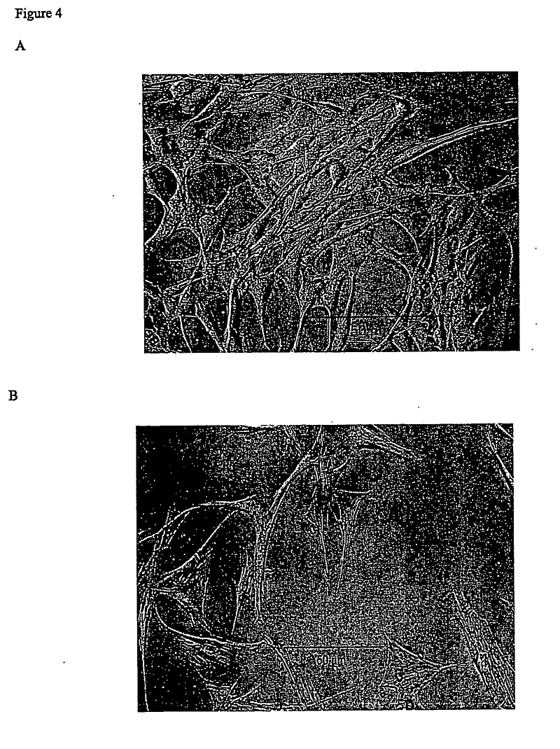

FIG. 4 illustrates the appearance of fibroblasts cultured on 100% acrylic acid plasma polymerised surface for 3 days with (A) and without (B) serum, 10% foetal calf serum (FCS);

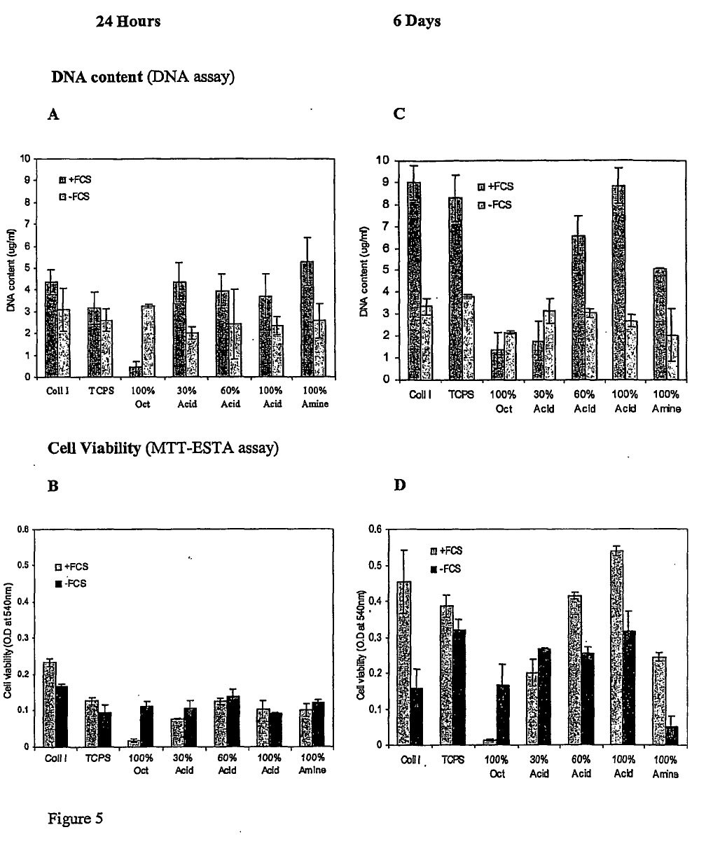

FIG. 5 illustrates the attachment of keratinocytes after 24 hours (A, B) and six days (C, D) to surfaces as shown by MTT-ESTA assay (A, C) and DNA assay (B, D). All cells were freshly isolated (seeded at 3.8×10⁵ cells/ml). Results shown are the means +/− standard deviation of triplicate wells of cells;

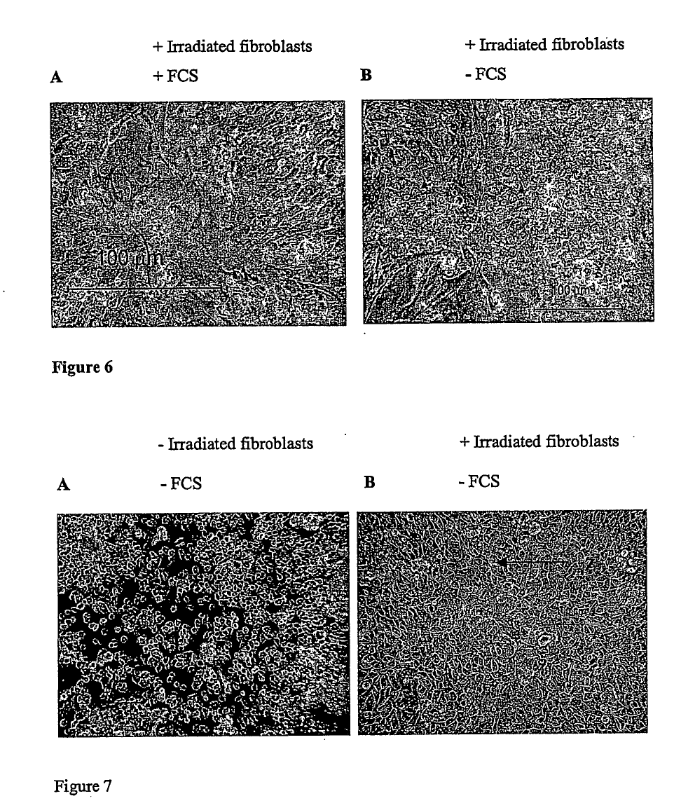

FIG. 6 illustrates the effect of serum on the co-culture of keratinocytes on a fibroblast feeder layer on a pure 10W acrylic acid surface at day 3, picture A with serum and picture B without;

FIG. 7 illustrates the influence of a fibroblast feeder layer on keratinocyte culture on a 10W acrylic acid surface in the absence of serum. Cells are cultured without serum in the absence (A) or presence (B) of a fibroblast feeder layer for seven days. Arrow X shows a typical differentiated cell and Y points to a region of golden unattached cells; in B a healthy confluent sheet of cells has formed with well-defined boundaries Z, in the presence of a feeder layer.

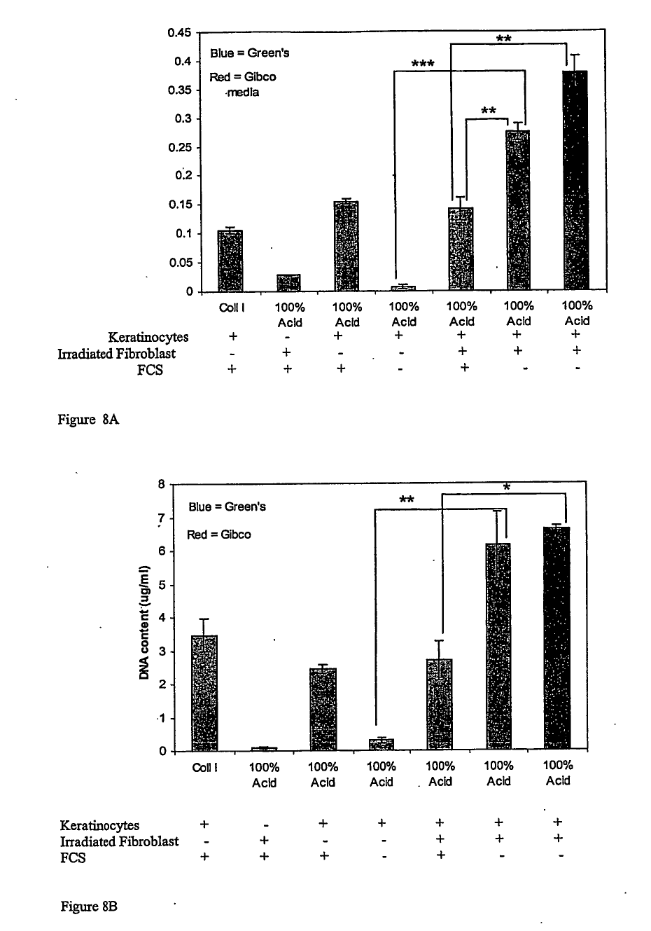

FIG. 8 illustrates the effect of serum and irradiated fibroblasts on the proliferation of keratinocytes on 10W pure acrylic acid. Cells are cultured on surfaces alone and in co-culture for seven days. (A) shows MTT-ESTA values and (B) DNA values. Values shown are means +/− standard deviation of n=3 triplicate wells. Values differing significantly from each other are indicated by * p<0.05, ** p<0.01 and *** p<0.005; and

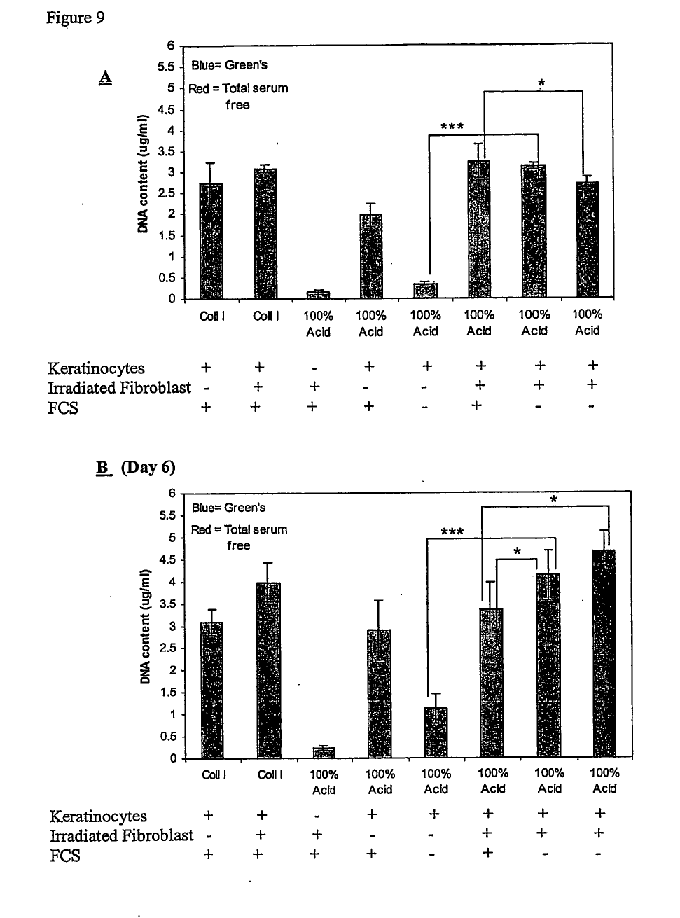

FIG. 9 is a DNA assay illustrating the effect of serum on the proliferation of both keratinocytes and irradiated fibroblasts, separately and in co-culture at day 4 (Graph A) and day 6 (Graph B). Values shown are means +/− standard deviation of n=3 triplicate wells. Values differing significantly from each other are indicated by * p <0.05, ** p<0.01 and *** p<0.005;

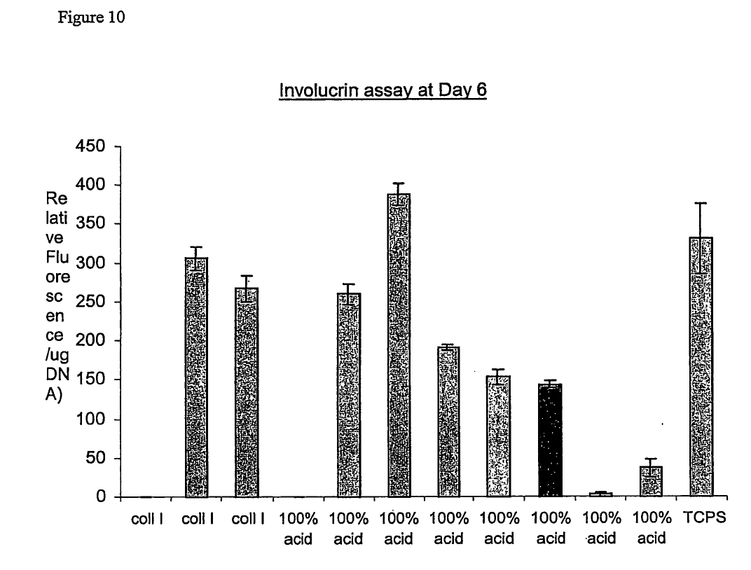

FIG. 10 illustrates quantitative data on the amount of involucrin expressed by keratinocytes at day 6 of co-culure which showed that without serum the keratinocytes expressed less involucrin at this timepoint;

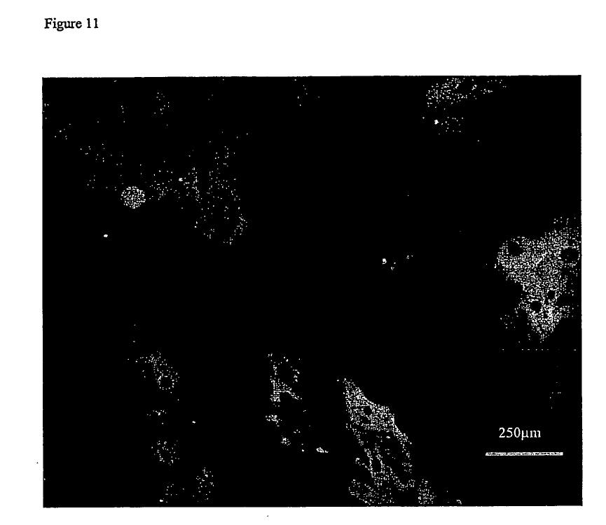

FIG. 11 illustrates irmmunofluoresence of the amount of involucrin expressed by keratinocytes under serum free co-culture at day 6; and

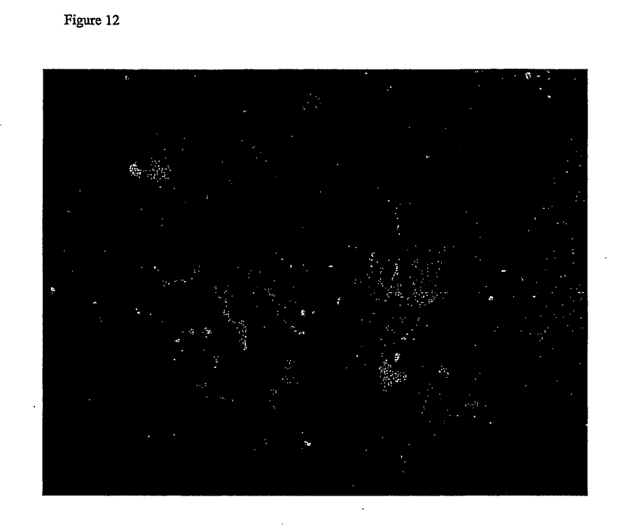

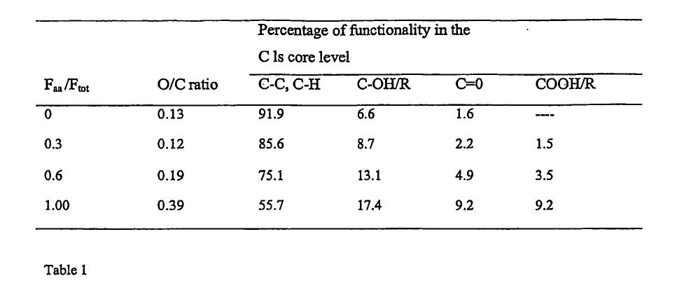

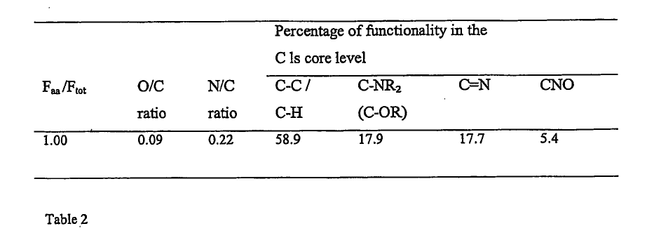

FIG. 12 illustrates immunofluoresence the greater amount of involucrin expressed by keratinocytes at day 6 under co-culture with serum present.

MATERIALS AND METHODS

Plasma Co-Polymerisation

Acrylic acid (>99%), octa-1,7-diene (>99%) and allyl amine (>99%) were obtained from Aldrich Chemical Co (UK). They were used as received following several freeze-pump/thaw cycles. Polymerisation was carried out in a cylindrical reactor, connected to a vacuum and liquid nitrogen pump. The plasma was sustained by a radio-frequency signal generator (13.56 MHz) and amplifier inductively coupled via an impedance matching unit and an externally wound copper coil. Monomers were either polymerised or co-polymerised at a plasma power of 2W or 10W at a total flow rate of 2.0 cm³₍ₛₜₚ₎ min⁻¹. A matching network ensured that power loss was minimal, the forward power was kept to a maximum and the reflected power was reduced to the lowest possible level. Monomer flow rate was calculated by converting the pressure change measured in the plasma reactor using a method described by Yasuda, which assumes ideal gas behaviour [12]. The pressure in the reactor was typically 3×10⁻²bar during polymerisation. Plasma polymers were deposited onto clean aluminium foil coated glass cover slips for XPS analysis; on to clean glass slides for contact angle measurements and on to tissue culture well plates (TCPS) for cell culture work. Substrates were placed in an identical position in the reactor for each experiment to avoid any variations in plasma deposition through the reactor. A deposition time of twenty minutes was sufficient to deposit a plasma coating with sufficient thickness to mask any substrate signal from the XPS spectrum. In addition, the monomer mixtures were allowed to flow for a further 15 minutes after the plasma had been turned off. This helped to minimise the uptake of atmospheric oxygen by the coatings upon exposure to the atmosphere [9]. Inn addition, the flow rate was checked at the end of the experiment to check that no leaks had occurred during plasma polymerisation.

X-ray Photoelectron Spectroscopy (XPS) Analysis

The coating deposited on the aluminium foil was analysed using X-ray photoelectron spectroscopy (XPS) 30 day after plasma polymerisation to allow for reported ageing of the samples, most notably in allyl amine and octadiene plasma coated substrates [13]. XPS was performed using a VG CLAM 2 spectrometer with Mg Kα X-ray source operating at a power of 100W. The spectrometer was calibrated using the Au 4f 7/2 peak position at 84.00 eV and the separation between the C 1s and F 1s peak positions in a sample of PTFE measured at 397.2 eV, which compares well with the value of 397.19 eV reported by Beamson and Briggs [14]. Spectra were acquired using a fixed take off angle of 30° with respect to the sample surface using Spectra 6.0 software (R. Unwin Software, Cheshire, UK). A wide scan (0-1100 eV) and narrow scans of each sample were acquired. Wide scans were used to obtain the surface oxygen/carbon (O/C) ratio and the narrow scans used to obtain information on the carbon, oxygen and nitrogen binding environments. For the collection of spectra for the wide and narrow scan, the analyser pass energies used were 50 and 20 eV respectively.

ESCA300 (Scienta Software) was used to obtain the peak fits of the C1s core level spectra. Gaussian-Lorenzian (G/L) peaks of mix 0.8-0.9 were fitted to the C 1s core level spectrum using well-established chemical shifts [15]. In the peak fitting, the full width half maximums (FWHM) of the peaks were kept equal and in the range of 1.38 to 1.67. A hydrocarbon peak was set to 285 eV to correct for any sample charging. Sample charge was in the region of 4-5 eV.

Contact Angle Measurements

Plasma polymer films were also deposited onto glass covers slips in order to examine the wettability of the surfaces and their stability to dissolution by using contact angle measurements. Contact angle measurements are frequently used to monitor the change in the concentration of polar and non-polar groups at the outermost 0.5-1.0 mm of the surface. A Rame-Hart goniometer (model 100-00(220)) from Burge Equipment, UK was employed. All contact angle recordings were carried out as described in full by this laboratory previously [16] and in accordance with criteria laid out by Andrade [17]. Contact angle measurements were taken in both advancing and receding modes by adding and removing 4 μl increments of distilled water, up to and including 20 μl. Advancing angles are representative of the low-energy part of the surface and receding angles are more characteristic of the high-energy part. At least 3 measurements were taken for each sample surface.

Cell Culture

Human dermal fibroblasts were obtained from the dermal layer of the skin after trypsinisation of a split-thickness skin graft, which was taken from specimens following routine surgery procedures (breast reduction and abdominoplasty), following washing in PBS and then minced finely with a scalpel and placed in 0.5% collagenase. Following centrifugation of the collagenase digest and elimination of the supernatant, the cells were resuspended in 10 mls of fibroblast culture medium (FCM) in a T25 Flask. The flask is maintained at 37° C. in a 5% CO₂ atmosphere.

Every 500 ml of FCM consists of 438.75 mls of Dulbecco's Modified Eagle's medium (DMEM), 50 mls of Foetal Calf Serum (FCS), 5 mls of 1-Glutaimine, 5 mls of Penicillin/Steptomycin (10,000 U/ml and 10,000 ug/ml respectively), 1.25 mls of Fungizone. FCM without FCS contains an additional 50 mls DMEM to compensate. Fibroblast cells were passaged when 90-100% confluent and used between passage numbers 5 and 9. While comparing the attachment of fibroblasts to 2W and 10W plasma polymer with and without FCS, the same flask and passage number of cells was employed. Passaging of the fibroblasts was achieved using 1.5 ml of a 1:1 mixture of 0.1% trypsin and 0.02% EDTA per T25 flask. The cells required for the 10W experiment were frozen at the beginning of the 2W round of experiments in a cryovial to −80° C. in a 1 ml solution containing 0.9 ml of DMEM and 0.1 ml of DMSO, a cryoprotectant.

Human epidermal keratinocytes (obtained from breast reductions and abdominoplasties) were freshly isolated from the dermal/epidermal junction. Cells were cultured in Green's media, which included cholera toxin (0.1 nM), hydrocortisone (0.4 μgm⁻¹), EGF (10 ngm⁻¹), adenine (1.8×10⁻⁴ M), tri-iodo-L-thyronine (2×10⁻⁷ M), fungizone (0.625 μg ml⁻¹), penicillin (1000 IU ml⁻¹), streptomycin (1000 μg ml⁻¹) and (optionally) 10% foetal calf serum. Cells were cultured at 37° C. in a 5% CO₂ atmosphere. Collagen coated TCPS well plates were prepared by air-drying a solution of collagen I (32 μg cm⁻²) in 0.1 M acetic acid (200 ug ml⁻¹) in a laminar flow cabinet overnight.

Assessment of Cell Attachment, Viability and Proliferation

For investigation of human dermal fibroblast attachment and viability, cells were seeded at a density of 7.0×10³ cells ml⁻¹ into 24 separate well plates (1.6 cm diameter). Human epidermal keratinocytes were seeded at a density of 3.8×10⁵ cells/ml. Co-culture experiments used a keratinocyte seeding density of 1.5×10⁵ cells/ml with irradiated dermal fibroblasts at 2×10⁴ cells/ml, irradiated for 4780 seconds using a Caesium 137 sealed source. The plates were plasma polymerised apart from the positive control of TCPS. The attachment and viability of the fibroblasts at three and seven days were assessed using an MTT-ESTA assay. This assay indicates viable cells and provides an indirect reflection of cell number, in that the cellular de-hydrogenase activity, which converts the MTT substrate to a coloured formazan product, normally relates to cell number. Cells were washed with 1 ml of PBS solution and then incubated with 0.5 mg ml⁻¹ of MTT in PBS for 40 minutes. 300 μl of acidified Isopropanol was then used to elute the stain. 150 μl was then transferred to a 96 well plate. The optical density was read using a plate reader set at a wavelength of 540 nm with a protein reference of 630 nm subtracted. In addition, the appearance of the cells was assessed and recorded at three and six or seven days.

The DNA content of the cells (which reflects cell number but not necessarily viability) was calculated at the same time periods using a Hoechst fluorescent stain (33258 Sigma Chemicals). Cells were incubated in 1 ml of digestion buffer for 1 hour. This buffer consisted of 48 g urea, which breaks up the cells and 0.04 g of Sodium Dodecyl Sulfate (SDS), which protects the cells from DNAase, per 100 ml of saline sodium citrate (SSC). Following digestion, cells were stained using the Hoechst fluorescent stain, in an SSC buffer at 1 μg/ml. A fluorimeter was used to measure the fluorescence using excitation and emission wavelengths of 355 and 460 nm respectively. A standard curve of known DNA concentrations was used to calculate the DNA content. For all experimental data presented, cells cultured on their own or in co-culture for six or seven days had a fresh change of media at day three.

Statistics

The significance of an irradiated fibroblast feeder layer in improving keratinocyte proliferation with and without serum was analysed using a statistical two-tailed Student t test where values of p<0.05 were considered as statistically significant.

Assessment of Differentiation

Keratinocyte differentiation in cell culture and co-cultures with and without serum was assessed by Involucrin expression, by fixing and staining using a commercially available antibody. Involucrin expression in the presence/absence of serum was determined by visual examination and quantitative measurement by eluting the fluorescene with sodium hydroxide digest (18). The fluorescence was quantified by relating it to the DNA content of parallel cultures.

Transfer Experiments

Transfer of keratinocytes, co-cultured as above with fibroblasts, in the absence of serum, from a culture surface (plasma polymerised acrylic acid deposited onto a PVC polymer film—Solmed) was assessed using an in-vitro wound bed model, comprising a de-epidermalised dermis (DED). Full details of the experiment and materials used are given in ref. 10

Co-Culture without Lethal Irradiation of Fibroblasts

In the absence of serum, non-irradiated fibroblasts were used to support keratinocyte culture on a 10W acrylic acid plasma polymerised surface. Expansion of keratinocytes was carried out in a keratinocyte friendly media (Green's), using a minimum seeding density of keratinocytes and fibroblasts of 1×10⁵ keratinocytes and 5×10⁴ fibroblasts per well [in 24 well tissue culture plate (well area=133 mm²)]. Alternatively, using non-irradiated fibroblasts, keratinocyte culture was supported using a keratinocyte seeding density of 5×10⁵ keratinocytes with 5×10⁴ fibroblasts per well.

### EXAMPLE 1

Characterisation of Plasma Co-Polymers.

Four plasma co-polymer surfaces were prepared from acrylic acid and oct-1,7-diene and one from allyl amine at both 2W and 10W powers. Plasma polymers are defined the % acrylic acid in the monomer flow (and we continue with this convention in the Examples, unless otherwise stated). The hydrocarbon diluent, octa-1,7-diene, allowed control of the resulting functional group concentration by promoting cross-linking of the acrylic acid monomer. XPS analyses of all plasma polymer surfaces deposited onto aluminium foil from acrylic acid and oct-1,7-diene revealed only carbon and oxygen. As expected, the O/C ratios increased as the molar fraction of acrylic acid from the monomer was increased. Plasma polymers containing octa-1,7-diene inevitably incorporated oxygen into the film upon exposure to the atmosphere prior to XPS analysis. XPS wide scans of plasma polymerised allyl amine surfaces revealed carbon, nitrogen and oxygen in the deposits. It is reasonable to assume incorporation of oxygen from the atmosphere may have occurred along with oxygen from the plasma. In addition, water is thought to desorb from the lining of the vessel walls within the reactor [9].

The C1s spectra were peak fitted using a range of oxygen containing functionalities [14]. For acrylic acid polymerisations, alcohol/ether (C—OH/R) groups were fitted at +1.5 eV, relative to the hydrocarbon peak (C—C/H), carbonyl (C=0) at +3.0 eV, carboxyl/carboxylate (COOH/R) at +4.0 eV and finally a β-shifted carbon, relative to the hydrocarbon at +0.7 eV. For octadiene, COOH/R and a β-shift were not fitted. In the peak fit, the ether functionality is counted twice since two carbon atoms experience the same shift brought about by one shared oxygen atom. While XPS cannot distinguish between ester and carboxylic acid groups, Alexander and Duc employed tri-fluoro ethanol (TFE) to label acids in plasma-polymerised acrylic acids and have shown that at similar power/flow ratios to those employed in this study, approximately 30% of the carboxylate peak can be assigned to acid [19]. O'Toole et al concluded using grazing angle IR spectroscopy that 10W fabricated acrylic acid samples contained a greater proportion of ester rather than acid groups. Conversely, at low powers (<5W), the entire COOH/R may be assigned to the carboxylic acid functional group [19b]. Based upon these studies, we acknowledge that not all of the COOH/R group will be acid. The correlation between the proportion of acrylic acid in the monomer feed and the resulting concentration of carboxylate groups in the plasma polymer is shown in Table 1 for 10W surfaces. A narrow scan spectra for a pure acrylic acid plasma polymer fabricated at 10W is illustrated in FIG. 1. No data are provided for 2W polymerisations.

C1s core level fits for allyl amine were peak fitted for nitrogen-containing functionalities using chemical shift values reported from the literature [14, 20]. The XPS data were quantified and are shown in Table 2. Functionalities fitted were imine (C═N) at 0.9 eV, amine (C—NR₂) at 1.7 eV and amide (CNO) at 3.0 eV. However, the nature of the amine (primary, secondary or tertiary) is unknown. Errors arising from peak fitting and in the measurement of peak areas used to determine surface elemental concentrations are generally considered to be +/−5% [14]. In the calculation of both the 0/C and N/C ratio from the peak fit, the total number of carbons bonded to either the oxygen or nitrogen was divided by 100 carbon atoms to yield respective O/C and N/C ratios.

### EXAMPLE 2

Stability of Surfaces

Advancing and receding contact angle measurements were recorded on all 2W and 10W plasma polymerised glass slides following immersion in water 0, 1, and 24 hours at 37° C. in a 5% CO₂ atmosphere. The relative hydrophobic/hydrophilic nature of each film was noted with the addition and removal of 4 μl increments of distilled water. With the 2W plasma polymerised surfaces there was considerable variation in the contact angle measurements—often the surfaces would visibly detach from the glass cover slip following immersion in water and these were therefore unsuitable for cell culture purposes (data not shown). In contrast the 10W surfaces were very stable to immersion in water for 24 hours as illustrated in FIG. 2. The most hydrophobic 10W surface was the pure hydrocarbon octadiene (89°) and the most hydrophilic was the pure acrylic acid surface (46°). FIG. 2 shows an example of a pure acrylic acid 10W surface stable to dissolution over 24 hours. The other 10W surfaces shared a similar stability to dissolution. From these results the decision was made to continue work with 10W rather than 2W surfaces.

### EXAMTPLE 3

Fibroblast Culture on plasma Co-Polymers

The attachment and proliferation of human dermal fibroblasts on 10W plasma surfaces was examined using MTT and DNA assays at three and seven days culturing cells with and without Foetal Calf Serum (FCS) (See FIG. 3). Photographs were also taken to record the morphology of the cells (FIG. 4). Similar results were obtained whether cell behaviour was assessed by DNA assay or MTT assay for cell viability. At three days and seven days cells performed poorly on the 100% octadiene surface but well on all other surfaces whether tissue culture plastic or surfaces prepared with gas flows containing 30, 60 or 100% acrylic acid or allyl am,ine. Cells clearly performed better in the presence of serum than in its absence and this was much more evident by day seven (considerable cell proliferation would have taken place by day seven but relatively little at day three). This is also evident in the appearance of the fibroblasts seen in FIG. 4, where on 100% acrylic acid at three days cells in the presence of serum were well organised and in greater number (FIG. 4A), but in the absence of serum cells were clearly fewer in number with relatively abnormal cytoskeletal arrangement (FIG. 4B) compared to fibroblasts in the presence of serum.

### EXAMPLE 4

Keratinocyte Culture on Plasma Co-Polymers.

In assessing keratinocyte performance on these surfaces, two internal controls were used, tissue culture plastic (TCPS) and collagen I which keratinocytes attach to readily. Freshly isolated keratinocytes were seeded at 3.8×10⁵ cells/ml on all surfaces in 24 well plates. Keratinocytes were cultured in standard Green's media in the presence and absence of foetal calf serum. Keratinocytes cultured without FCS in the medium had attached well after 24 hours (FIG. 5 A, B) but were less able to proliferate in the absence of serum as was evident by six days (FIG. 5 C, D). Here in contrast to the fibroblasts, which attached and proliferated equally well on all surfaces, the percentage of acrylic acid in the monomer feed had a significant effect on the level of keratinocyte attachment irrespective of whether this was assessed by MTT or DNA assay. Again, generally the level of cell attachment and proliferation as estimated by the MTT-ESTA assay, by day six mirrored that shown for the DNA assay (FIG. 5). On the pure acrylic acid surface containing 9.2% COOH/R, the level of keratinocyte proliferation in serum containing media was greatest and comparable to that measured on collagen I (FIG. 5 C, D). Throughout all experiments the cells performed poorly on the 100% octadiene surface.

### EXAMPLE 5

Co-culture of Human Dermal Fibroblasts and Epidermal Keratinocytes on 100% Acrylic Acid Surface Fabricated at 10W

The surface selected for co-culture of both cells was the acrylic acid surface (prepared from 100% acrylic acid) as this provided the best surface for attachment and proliferation for the keratinocytes and as fibroblasts also performed well on this surface (as they did on surfaces fabricated using lower percentages of acrylic acid in the monomer flow). This 10W surface was then chosen to investigate co-culture conditions for these two cell types exploring culture of keratinocytes in the presence and absence of foetal calf serum. As a substitute for foetal calf serum irradiated fibroblasts were used. The irradiated fibroblasts were initially seeded at 2×10⁴ cells/ml for 24 hours in DMEM in the presence of serum. Thereafter, for any co-culture investigation, the media was removed and replaced with keratinocytes seeded at 1.5×10⁵ cells/ml either with or without serum. Keratinocytes (with and without serum in the media) and irradiated fibroblasts were also cultured on their own for comparative purposes. A positive control of Collagen I was employed throughout. By three days, the keratinocytes had formed colonies well in the presence of an irradiated dermal feeder layer both with and without serum in the Green's media (FIG. 6). However, by day seven, keratinocytes cultured without irradiated fibroblasts under serum free conditions had started to detach from the surface (FIG. 7A). In contrast, cells in serum-free media in the presence of an irradiated fibroblasts (FIG. 7B) had formed a confluent healthy sheet of keratinocytes and were starting to form multilayers.

Quantitative data on the contribution of the fibroblast feeder layer under serum free conditions is shown in experiments summarised in FIGS. 8 and 9. The MTT-ESTA and DNA data in FIG. 8 illustrates the improved performance of the keratinocytes both in terms of attachment and proliferation in the presence of an irradiated fibroblast feeder layer under serum free co-culture conditions after seven days of co-culture Co-culture of cells was also explored in a commercially available defined media (Gibco defined media), which has been designed to optimise the proliferation of keratinocytes. As can be seen, both MTT and DNA values clearly indicate that in the absence of serum there is considerable synergy between the irradiated fibroblasts (which contribute negligible DNA themselves) and the keratinocytes (which on their own do badly in the absence of serum). Co-culture of cells in Gibco medium (no serum) also did very well compared to cells in serum containing conditions on the 100% acid surface or cells on collagen I.

Further steps were taken to exclude serum contamination from fibroblast cultures by irradiating fibroblasts in serum-free media and then maintaining them with the keratinocytes, serum free for the entirety of the experiment. When both cells were combined serum free on the 100% acrylic acid surface then DNA values equivalent to that seen in the presence of serum were achieved at day four and day six (FIG. 9 A, B). When keratinocytes were cultured on 100% acrylic acid or on collagen I in the presence of serum, the addition of a fibroblast feeder layer made little difference; in contrast however in the absence of serum, where keratinocytes on 100% acrylic acid performed relatively poorly, the addition of a fibroblast feeder layer dramatically improved keratinocyte proliferation (as also illustrated in FIGS. 7 A, B). Comparing the results at day 4 with those at day 6 (FIGS. 9 A, B), it is clear that cultures continued to proliferate. Similar results were obtained whether cells were cultured in Green's media serum free or in Gibco serum free media

Keratinocytes cultured on the plasma polymer surface in the presence of irradiated fibroblasts but the absence of serum showed less differentiation, as assessed by measuring involucrin expression relative to DNA content of the cells than those cultured in the presence of serum, whether on the plasma polymer surface or on collagen I. Involucrin was measured as a component of the qualified envelope produced by keratinocytes as they begin to differentiate.

Experiments were conducted using the protocol as previously described and then cultures were fixed and stained for involucrin using a commercially available antibody and cells photographed at this stage. Keratinocytes cultured in the absence of serum showed less involucrin expression per culture than those cultured with serum. This result was confirmed quantitatively by then eluting the fluorescence with sodium hydroxide digestion (full method as in Little et al, 18) and the fluorescence quantified by relating it to the DNA content of parallel cultures.

In addition to the above results obtained culturing keratinocytes in Green's media, the use of keratinocyte defined media (which has 1/10th of physiological calcium) gave keratinocytes with a low expression of involucrin (as physiological calcium is required for keratinocyte differentiation). Keratinocytes cultured in keratinocyte defined medium on the plasma polymer surface also showed a very low expression of involucrin both with and without irradiated fibroblasts. Thus keratinocytes in Green's and keratinocytes in keratinocyte defined medium do well on this surface in terms of proliferation and a low degree of differentiation.

The purpose of this study was to develop a surface for culture and transfer of keratinocytes which could be used clinically and which might have advantages over other approaches for delivering keratinocytes to patients' wounds. Of the methodologies currently available, expansion of keratinocytes on a lethally irradiated layer of mouse fibroblasts and then detaching these cells as an integrated sheet of cells using trypsin is the current “gold standard” (Rheinwald and Green [1]). This method has been in use since the early 1980s and has been used in the treatment of patients with extensive skin loss due to burns injuries in Europe and the USA. Unfortunately, its acknowledged that the clinical “take” of these cultured skin grafts is generally less than 50% overall [2,3]. This may be due to a combination of factors, including the condition of the patient's wound bed. However, there are two issues of cell culture which no doubt contribute to this poor take—one is that cells are detached enzymically and this can cause damage to the cell surface receptors (integrins) which keratinocytes require for adhesion to the wound bed. The second issue is that in order to form an integrated sheet of cells appropriate for attachment, cells are cultured until they are well advanced in terms of differentiation. Such cells are less likely to perform well on a wound bed. It was against this background, that we developed a plasma polymer surface for the culture of keratinocytes and transfer of cells to wound bed, which avoided the need to use trypsin to detach the cells and which did not require cells to form a confluent sheet.

The current study seeks to develop this work further by introducing a fibroblast feeder layer to aid the expansion of keratinocytes on this surface, to help maintain keratinocytes in a proliferative phenotype and, as part of this approach, to explore the possibility of developing a culture system that could be serum free.

The main findings of this study are that it is possible to get as rapid an expansion of keratinocytes on a 10W plasma polymer surface containing 9.2% COOH/R groups under serum free conditions if a feeder layer of fibroblasts is present as can be obtained by the current “gold standard” method of culturing cells on irradiated mouse fibroblasts with foetal calf serum present. Cells can be cultured successfully on a surface coated with collagen I and have indeed been used clinically, however, sources of collagen I are usually bovine collagen [4]. A concern throughout Europe currently is that BSE cannot be detected by any in vitro tests and therefore it is impossible to be confident that bovine material is BSE free unless it comes from herds that have never been exposed to BSE. While this is not viewed as a major concern in the US, European regulatory authorities would prefer that cells that are cultured for clinical use avoid the use of bovine, and indeed other animal-derived products, to reduce the risk of disease transmission. Thus, a ‘holy grail’ of cell culture for clinical use is to develop an entirely defined culture system. The work presented in this study is an important step towards that goal.

We shall first of all discuss the nature of the surface and then the behaviour of the cells on the surface. Plasma polymerisation of acrylic acid and oct-1,7-diene produced a wide range of plasma polymers with varying concentrations of carboxyl /carboxylate groups (COOH/R). The XPS data showed a linear relationship between the O/C ratio and the fraction of acrylic acid in the monomer feed. Contact angle measurement results illustrated the unstable nature of the 2W plasma polymer films and their susceptibility to peel away from the glass slide following submersion in distilled water. A previous study from this group demonstrated this problem with low power films and overcame it by incorporating of octa-1,7-diene into the monomer feed, providing cross-linking with the acrylic acid and improving the stability of the surface [16]. Alternatively, by increasing the power to 10W it was possible to improve the stability of the surface as illustrated by FIG. 2 and this option was chosen for these studies. A downside to employing a higher power was the increased fragmentation of the monomer resulting in a greater range of oxygen-carbon functionalities, making it more difficult to ascribe the exact percentage of acid groups present.

Human dermal fibroblasts were initially successfully cultured on acrylic acid and allyl amine based plasma polymer surfaces with FCS in the media. Without serum the fibroblasts were unable to proliferate, this was evident after 72 hours of culture. Throughout the experiment, the 100% octadiene surface acted as a negative control, indicating the significance of the surface chemistry of both the acrylic acid and allyl amine surfaces in providing attachment for the human dermal fibroblast cells. Greisser et al have noted the affinity of negatively charged proteins to the positive charge provided by protonated amine groups (amide groups) at physiological pH 7.4 [21]. Although there is no reported work on the culture of human dermal fibroblast cells on acrylic acid based plasma polymer surfaces, many studies have noted that fibroblasts spread on a wide range of higher surface energy surfaces [22, 23]. The MTT and DNA results for the culture of human dermal fibroblasts on 10W surfaces support both of these statements. Good cell attachment was witnessed on the 100% acrylic acid surface at 10W, containing 9.2% COOH/R groups but good attachment was also seen on lower percentage acrylic acid surfaces as well, containing less than 9.2% COOH/R groups and the pure allyl amine surface. The advancing and receding contact angle measurements, monitoring the stability of this surface to dissolution further emphasised the suitability of these 10W plasma polymers for cell culture applications.

The attachment of human epidermal keratinocytes to plasma polymer surfaces was clearly best on the 100% acrylic acid surface fabricated at 10W. An increase in the amount of acrylic acid in the plasma polymer led to an increase in proliferation of keratinocytes. Cell attachment to this surface was comparable to the cell attachment to collagen I, a well-established substratum material for culturing keratinocytes. The performance of the keratinocytes serum-free was better than the fibroblasts, but it is important to note the higher initial cell density employed in this experiment (3.8×10⁵ cells/ml) compared to fibroblasts cell density of 7×10³ cells/ml. This was to account and compensate for the high percentage of already differentiated keratinocytes, which occur following isolation of the freshly isolated keratinocytes from the epidermal/dermal junction. Previous studies examining cellular attachment to plasma polymer surfaces have noted optimal keratinocyte attachment on 2W pure acrylic acid surfaces, containing 2.3% carboxylic acid groups. Due to the greater degree of fragmentation of the monomer in the plasma at 10W (c.f. 2W), the relative contribution of acid to the COOH/R peaks is unknown but certainly less than 100% not unreasonable to assume that <50% of COOH/R is carboxyl (≦4.6%) [19b].

Co-culture of keratinocytes with fibroblasts clearly illustrated the benefit of an irradiated human dermal fibroblast layer in enhancing the performance of the keratinocyte in the absence of serum. When irradiated fibroblasts and keratinocytes were co-cultured with serum the contribution of the fibroblast to keratinocyte proliferation was marginal compared to their contribution in serum free data. It appears that the feeder layer provides all the necessary growth factors and extracellular matrix proteins sufficient to support serum free keratinocyte growth. This supports and extends the observation initially reported by Rheinwald and Green using a 3T3 cell line under serum containing conditions [1].

The major benefit in avoiding the use of serum currently lies in avoiding problems associated with detecting BSE. For this reason we used human rather than mouse fibroblasts in this study. (In this study human fibroblasts were initially expanded in media with sera and then cultured under serum-free conditions. For clinical use it will be necessary to expand fibroblasts from the initial patient biopsy using serum-free defined media containing recombinant mitogens). In considering how cells attach to a surface it was interesting to note that both fibroblast and keratinocytes attached successfully to the 10W plasma polymer surface under serum free conditions (although neither proliferate well in the absence of serum). Attachment of cells could be occurring directly to the surface or through a protein layer attached to the surface [9]. When serum is present the protein coating the surface will be serum derived; in its absence the cells themselves may secrete the proteins. A typical example is fibroblasts, which secrete large amounts of fibronectin in culture (as outlined in Ralston et al [7]). Serum in contrast contains both adhesive (fibronectin and vitronectin) and anti-adhesive proteins (e.g. very large amounts of albumin). Serum also contains a range of platelet-derived mitogens such as platelet-derived growth factor (PDGF), epidermal growth factor (EGF) and transforming growth factor (TGF-β) which all stimulate cell proliferation [25]. It is because of these mnitogens that serum is extensively used in cell culture. In producing defined media recombinant mitogens are used.

The aim of this study was to develop improved methodology for the expansion and transfer of mammalian cells, in the example, keratinocytes, for clinical wound healing. We sought to culture cells on a surface appropriate for cell delivery to wound beds while improving the rate of keratinocyte expansion and developing a culture system which did not require the use of xenobiotic materials. The approach we took was to culture keratinocytes on a plasma-polymerised copolymer introducing growth arrested human dermal fibroblasts as a source of mitogens for keratinocyte expansion. Plasma copolymers of acrylic acid/octa-1,7-diene and allyl amine were prepared and characterised using X-ray photoelectron spectroscopy (XPS). Polymers were fabricated at 2W and 10W. 10W surfaces proved more stable than 2W surfaces. Fibroblasts attached and proliferated well (in the presence of foetal calf serum) on all surfaces fabricated with 30-100% acrylic acid in the monomer flow. In contrast, keratinocytes proved more selective and only attached and proliferated on surfaces produced from 100% acrylic acid (giving a 9.2% carboxyl/carboxylate in the plasma deposite) with relatively poorer attachment with the addition of octdiene to the monomer flow, compared to attachment to collagen I. Attachment of fibroblasts and keratinocytes was not greatly affected by the omission of serum but serum was required for proliferation of both cells. However, using a pure acrylic acid surface, fabricated at power of 10W and a non-proliferative fibroblast feeder layer of human dermal fibroblasts, rapid expansion of human keratinocytes was achieved in entirely serum free conditions. The results obtained were as good as those obtained culturing keratinocytes and growth arrested fibroblasts on tissue culture plastic or on a substrate of collagen I in the presence of serum. Thus, by combining a chemically defined surface and growth-arrested fibroblasts, keratinocyte expansion can be achieved under serum free conditions. This culture system offers an attractive approach for the culture of keratinocytes for clinical use.

The potential of serum free co-culture of keratinocytes on plasma polymers is exciting. There lies the opportunity to utilise plasma-polymers as synthetic surfaces capable of acting as a cell delivery vehicle to wounds free of animal based products. The next stage in developing the serum-free co-culture system for clinical use will be to examine the performance of keratinocytes in the absence and presence of fibroblasts in transferring to an in vitro wound bed model (as recently performed from our laboratory) [10].

In summary the current study shows that the addition of irradiated human fibroblasts to the culture of human epidermal keratinocytes on a 10W plasma polymer surface containing 9.2% COOH/R groups allows accelerated keratinocyte proliferation under serum free conditions.

### EXAMPLE 6

Transfer of Keratinocytes from Plasma Polymer Surface to DED

Keratinocytes co-cultured in the absence of serum with fibroblasts on an acrylic acid plasma polymerised surface successfully transfered to a DED surface.

### EXAMPLE 7

Co-culture without Lethally Irraditating Fibroblasts

The use of non-irradiated human fibroblasts (as opposed to lethally irradiated fibroblasts) in the culture of human epidermal keratinocytes on a 10W acrylic acid plasma polymer surface containing 9.2% COOH—R groups allows keratinocyte proliferation under serum free conditions. These results are shown in Table 3

TABLE 3
Time taken for keratinocytes to achieve 90% surface area after mixing
with human fibroblasts and co-seeding in serum free Green's media.
Results shown are means of triplicate

| Keratinocyte Density | Fibroblast Density | |
|---|---|---|
| (cells per well) | (cells per well) | 90% Confluence |
| 5 × 10⁵ | 1 × 10⁵ | 3-4 days |
| 5 × 10⁵ | 5 × 10⁴ | 3-5 days |
| 5 × 10⁵ | 1 × 10⁴ | 9 days |
| 1 × 10⁵ | 1 × 10⁵ | 7 days |
| 1 × 10⁵ | 5 × 10⁴ | 8 days |
| 1 × 10⁵ | 1 × 10⁴ | Not achieved |
| 5 × 10⁴ | 1 × 10⁵-1 × 10⁴ | Not achieved |

These results show successful expansion of keratinocytes requires that one uses a keratinocyte friendly media such as Green's and a minimum seeding density of keratinocytes—the minimum density of keratinocytes was 1×10⁵ keratinocytes per well (bottom of well area=133 mm²). At seeding density of 1×10⁵ keratinocytes per well it required 5×10⁴ fibroblasts per well to achieve a surface which was 90% occupied by keratinocytes at 8 days. If only 1×10⁴ fibroblasts were used, keratinocytes did not achieve a significant surface area. However, if the experiment was begun with 5×10⁵ keratinocytes and 5×10⁴ fibroblasts then within 4 days more than 90% of the well surface was occupied by keratinocytes. Experiments looking at the ratio of keratinocytes to fibroblasts to achieve keratinocyte expansion showed that there was a minimum seeding density of keratinocytes per well (1×10⁵ per well). It was necessary to use a media which supported keratinocytes (if one used a media which supported fibroblasts this was not successful) and there needed to be a ratio of fibroblast to keratinocytes of ideally 1:1 at 1×10⁵ keratinocytes per well (per 133 mm²) and ratio not greater than ca. 5:1. Similar ratios can be determined for other keratinocyte seeding densities of greater than 1×10⁵ cells per well.

References

1. Rheinwald J, Green H, Serial cultivation of strains of human epidermal Keratinocytes: the formation of colonies from single cells, Cell, 1975, Vol 6, pp 331-344.

2. Boyce S, Design principles for composition and performance of cultured skin substitutes, Burns, 2001, Vol 27, pp 534-544.

3. Balasubrammi M et al, Skin substitutes: a review, Burns, 2001, Vol 27, pp 523-533.

4. Grant I et al, Demonstration of epidermal transfer from a polymer membrane using genetically marked porcine keratinocytes, Burns, 2001, 27, pp 1-8.

5. Boyce S, Ham R G, Calcium-regulated differentiation of normal epidermal keratinocytes in chemically defined clonal culture and serum-free serial culture, J Invest Dermatology, 1983, Vol 81, pp 33-40.

6. Wang H J et al, Human keratinocyte culture using porcine pituitary extract in serum-free medium, Bums, 1995,Vol 21, No 7, pp 503-506.

7. Ralston D et al, Keratinocytes contract human dermal extracellular matrix and reduce soluble fibronectin production in a skin composite, British Journal of Plastic Surgery, 1997, Vol 50, pp408-415.

8. Chakrabarty K et al, Development of autologous human dermal-epidermal composites based on sterilised human allodermis for clinical use, British Journal of Dermatology, 1999, Vol 41, pp 811-823.

9. France R et al, Attachment of human keratinocytes to plasma co-polymers of acrylic acid/octadiene and allyl amine/octadiene. Journal of Materials Chemistry, 1998. 8(1): pp 37-42.

10. Haddow D B et al, Plasma polymerised surfaces for culture of human keratinocytes and transfer of cells to an in vitro wound bed model, Journal of Biomedical Materials Research, 2003, 64, 80-87.

11. Haddow D B et al, Comparison of Proliferation and Growth of Human Keratinocytes on Plasma Co-polymers of Acrylic Acid/1,7 Octadiene and Self Assemble Monolayers, Journal of Biomedical Materials Research, 1999, 47(5), pp 379-387.

12. Yasuda H (1985) Plasma Polymerisation, Academic Press: New York, 1985, Chapter 3.

13. Whittle, J, R. Short, Differences in the ageing of Allyl Alcohol, Acrylic Acid, Allyl amine and octa-1,7-diene plasma polymers by XPS. Chem Mater, 2000. 12: p. 2664-2671.

14. Beamson, G. and G. Briggs, High resolution XPS of organic polymers: The Scienta ESCA300 Handbook. 1992: John Wiley and sons.

15. Hynes, A., Macromolecules, 1996. 29: p. 4220

16. Daw, R., Plasma Co-polymer Surfaces of acrylic acid/1,7 octadiene and Methyl Vinyl Ketone/Octa-1,7-diene: Surface, characterisation and behaviour of osteoblast like cells, in Engineering Materials. 1998, University of Sheffield: Sheffield.

17. Andrade J, Contact Angle and interface Energetics. Surface and Interfacial aspects of biomedical polymers. Vol. 2. 1985: New York and London. 249292.

18. Little M. C. et al, The Participation of Proliferative Keratinocytes in the Pre-Immune Response to Sensitizing Agents, British Journal of Dermatol., 1997, 138, 45-56

19. Alexander M, Duc, T, The chemistry of deposits formed from acrylic acid plasmas, J. Mater. Chem, 1998, 8(4), pp 937-943.

19b. O'Toole L, Beck, Short R, Characterisation of Plasma Polymers of Acrylic acid and Propanoic acid, Macromolecules, 1996, 29, pp 5172-5177.

20. Fally F, Quantification of the functional groups present at the surface of plasma polymers deposited from propylamine, allylamine, and propargylamine, J Appl. Polym. Sci, 1995, Vol 56, p 597-614.

21. Greisser H et al., Growth of human cells on plasma polymers: Putative role of amine and amide groups. J. Biomater. Sci. Polymer Edition, 1994. 5(6): p. 531-55412.

22. Altankov G et al, Studies on the biocompatibility of materials: Fibroblast organisation of substratum-bound fibronectin on surfaces varying in wettability. Journal of Biomaterials Research, 1996. 30: pp 385-391.

23. Ruardy T et al, Adhesion and spreading of human skin fibroblasts on physiochemically characterised gradient surfaces. Journal of Biomedical Materials Research, 1995. 29: pp 1415-1423.

24. Kelly J, Osteoblast Response to Oxygen Functionalised Plasma Polymer Surfaces, PhD thesis, 2001, University of Sheffield.

25. Freshney, R I, Culture of animal cells, Chapter 7 “The culture environment”. Wiley-Liss Inc. 1994, p74-p77.

1. A method for the culture of mammalian cells comprising the steps of:
i) providing a cell culture vessel comprising:
a) mammalian cells;
b) a cell culture support comprising a substrate wherein said substrate comprises a cell culture surface wherein said surface comprises a polymer of an acid monomer and attached thereto, fibroblast feeder cells;
c) cell culture medium sufficient to support the growth of said mammalian cells wherein said medium does not include serum;
ii) providing cell culture medium and conditions which promote the proliferation of said mammalian cells.

2. A method according to claim 1 wherein said mammalian cells are human.

3. A method according to claim 1 wherein said mammalian cells are maintained in culture in an un-differentiated state.

4. A method according to claim 1 wherein said mammalian cells are selected from the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

5. A method according to claim 4 wherein said mammalian cells are autologous keratinocytes.

6. A method according to claim 1 wherein the number of said mammalian cells and said fibroblast cells is at a ratio of about between 1:1-1:5.

7. A method according to claim 6 wherein said ratio is about 5:1.

8. A method according to claim 1 wherein said mammalian cells are seeded at about 0.75×10⁴ cells/mm².

9. A method according to claim 6 wherein said mammalian cells are keratinocytes.

10. A method according to claim 1 wherein said substrate comprises a non-porous polymer.

11. A method according to claim 1 wherein said substrate is a solid phase substrate.

12. A method according to claim 1 wherein said substrate is a porous material.

13. A method according to claim 12 wherein said material is a woven material.

14. A method according to claim 12 wherein said material is a non-woven material.

15. A method according to claim 1 wherein said cell culture surface comprises a polymer comprising an acid content of at least 2%.

16. A method according to claim 1 wherein said surface comprises a polymer comprising an acid content between about 2-20%.

17. A method according to claim 1 wherein said surface comprises a polymer comprising an acid content greater than 20%.

18. A method according to claim 15 wherein said polymer comprises an acrylic acid monomer with at least 2% acid content.

19. A method according to claim 18 wherein said acid content is between 2% and 10%.

20. A method according to claim 19 wherein said acid content is about 4-5%.

21. A method according to claim 1 wherein said polymer comprises an acid co-polymer.

22. A method according to claim 1 wherein said fibroblast feeder cells are non-proliferative.

23. A method according to claim 22 wherein said fibroblast feeder cells are rendered non-proliferative by lowering the calcium concentration of the growth medium.

24. A method according to claim 1 wherein said fibroblast feeder cells are human fibroblasts.

25. A method according to claim 24 wherein said fibroblasts are dermal or oral fibroblasts.

26. A method according to claim 24 wherein said fibroblasts are autologous.

27. A cell culture vessel comprising: a cell culture support comprising a substrate wherein said substrate comprises a cell culture surface wherein said surface comprises a polymer of an acid monomer and attached thereto, fibroblast feeder cells.

28. A vessel according to claim 27 wherein said vessel further comprises mammalian cells and cell culture medium which medium does not include serum.

29. A vessel according to claim 28 wherein said mammalian cells are selected from the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

30. A vessel according to claim 29 wherein said mammalian cells are keratinocytes.

31. A method to treat a cell culture vessel comprising the steps of:
i) providing at least one acid monomer source in a gas feed;
ii) creating a plasma of said acid monomer; and
iii) bringing into contact a cell culture vessel with said plasma monomer to provide a cell culture vessel comprising an acid polymer.

32. A method according to claim 31 wherein said acid monomer source comprises 30-99% acid monomer.

33. A method according to claim 31 wherein said acid monomer source consists of a 100% acid monomer source.

34. A method according to claim 33 wherein said acid monomer source consists of a 100% acrylic acid.

35. A method to treat a cell culture vessel comprising the steps of:
i) providing a selected ratio of an acid containing monomer and a hydrocarbon in a gas feed;
ii) creating a plasma of said mixture;
iii) bringing into contact a cell culture vessel with said plasma mixture to provide a cell culture surface comprising an acid co-polymer.

36. A method according to claim 35 wherein said plasma is created by means of electrical power input coupled by means of a copper coil or bands.

37. A method to culture mammalian cells on a therapeutic vehicle comprising the steps of:
i) providing a preparation comprising:
a) mammalian cells;
b) a therapeutic vehicle wherein said vehicle comprises a substrate which comprises a surface wherein said surface comprises a polymer of an acid monomer and attached thereto, fibroblast feeder cells;
c) cell culture medium sufficient to support the growth of said mammalian cells wherein said medium does not include serum; and
ii) providing cell culture conditions which promote the proliferation of said mammalian cells on said therapeutic vehicle.

38. A method according to claim 37 wherein said mammalian cells are human.

39. A method according to claim 37 wherein said mammalian cells are selected from the group consisting of: epidermal keratinocytes; dermal fibroblasts; adult skin stem cells; embryonic stem cells; melanocytes, corneal fibroblasts, corneal epithelial cells, corneal stem cells; intestinal mucosa fibroblasts, intestinal mucosa keratinocytes, oral mucosa fibroblasts, oral mucosa keratinocytes, urethral fibroblasts and epithelial cells, bladder fibroblasts and epithelial cells, neuronal glial cells and neural cells, hepatocyte stellate cells and epithelial cells.

40. A method according to claim 37 wherein said mammalian cells are autologous.

41. A method according to claim 39 wherein said mammalian cells are keratinocytes.

42. A method according to claim 37 wherein said fibroblast feeder cells are human.

43. A method according to claim 42 wherein said fibroblast feeder cells are dermal fibroblasts or human oral fibroblasts.

44. A method according to claim 42 wherein said feeder cells are autologous.

45. A method according to claim 37 wherein the number of said mammalian cells and said fibroblast cells is at a ratio of about between 1:1-5.1.

46. A method according to claim 45 wherein said ratio is about 5:1.

47. A method according to claim 45 wherein said mammalian cells are keratinocytes and are in a ratio of about 5:1 with said fibroblast cells.

48. A method according to claim 37 wherein said mammalian cells are seeded at about 0.75×10⁴ cells/mm².

49. A method according to claim 45 wherein said therapeutic vehicle comprises a substrate composed of a polymeric material wherein the ratio of mammalian cells to fibroblast cells is about 5:1.

50. A method according to claim 49 wherein said substrate is composed of a vinyl polymer.

51. A method according to claim 50 wherein said vinyl polymer is selected from the group consisting of: polyvinyl chloride, polyvinyl acetate, polyvinyl alcohol.

52. A therapeutic vehicle produced by the method according to claim 37.
